(19) United States
(12) Patent Application Publication
(10) Pub. No.: US 2013/0261984 A1
(43) Pub. Date: Oct. 3, 2013

(54) METHODS AND SYSTEMS FOR DETERMINING FETAL CHROMOSOMAL ABNORMALITIES

(71) Applicants: Michael A. Eberle, San Diego, CA (US); Thomas Royce, San Diego, CA (US)

(72) Inventors: Michael A. Eberle, San Diego, CA (US); Thomas Royce, San Diego, CA (US)

(73) Assignee: ILLUMINA, INC., San Diego, CA (US)

(21) Appl. No.: 13/791,543

(22) Filed: Mar. 8, 2013

Related U.S. Application Data
(60) Provisional application No. 61/618,425, filed on Mar. 30, 2012.

(51) Int. Cl. G06F 19/00; G06F 19/20; C12Q 1/68

(52) U.S. Cl. CPC G06F 19/345; C12Q 1/6874; G06F 19/20
USPC 702/20; 506/2; 702/19

(57) ABSTRACT

The present disclosure provides methods and systems for determining the presence or absence of aneuploidy in a fetus. In particular, the present disclosure provides noninvasive methods and systems for detecting the presence of fetal trisomy and other fetal chromosomal anomalies, paternity of a fetus and fetal genotype.

The present application claims priority to U.S. provisional patent application Ser. No. 61/618,425 filed Mar. 30, 2012, incorporated herein by reference in its entirety.

### BACKGROUND

Each person normally has 23 pairs of chromosomes, or 46 chromosomes (22 pair of autosomes and one pair of sex chromosomes). For each pair of chromosomes, one of the pair is inherited from the mother while the second chromosome of the pair is inherited from the father. However, due to errors distributing those parental chromosomes to a fetus, about 1 in 150 babies in the United States is born with a chromosomal abnormality. Children that inherit certain chromosomal abnormalities often are born with mental and/or physical birth defects, whereas other chromosomal abnormalities result in miscarriage or stillbirth.

When a chromosomal abnormality occurs, it is usually derived from an error that occurs when an egg or sperm cell develops. For example, one of the egg or sperm cells may divide incorrectly resulting in either too many or too few chromosomes. When these abnormal cells join with normal cells, the result is an embryo with too many or too few chromosomes; an embryo with a chromosomal abnormality.

A common type of chromosomal abnormality is a cell with three copies of a chromosome instead of the normal two copies. Three copies of a chromosome, or trisomy, is typical of individuals diagnosed with Down syndrome, where these individuals have three copies of chromosome 21.

However, chromosomal abnormalities do not only occur during fertilization; some chromosomal errors such as structural errors can occur prior to fertilization. Chromosomal structural errors can be, for example, due to parts of a chromosome that are duplicated, inverted, deleted or swapped with a part of another chromosome. Such structural defects may have no consequence, or they may have deleterious consequences to a fetus. Cell division errors of fertilized cells can also result in an abnormality called mosaicism where a child has different populations of cells with different genotypes.

The most common chromosomal abnormality is Down syndrome (also known as Down's syndrome, trisomy 2.1 and T21), which results from one too many copies of chromosome 21 and affects about 1 in 800 babies in the United States alone, the frequency increasing with a mother's age. Down syndrome is characterized by unique facial features along with mental and/or developmental challenges. Trisomy 13 or Patau Syndrome and trisomy 18 or Edwards Syndrome are less frequent than T21, but more severe oftentimes resulting in death of the infant prior to its first birthday. The Triple XXX genotype, wherein three X chromosomes are present in a girl's chromosomal complement, may go unnoticed, as do males with an extra Y, or XYY, chromosomal complement. However, males with an extra X Chromosome, or OXY (Klinefelter's Syndrome), may be infertile as adults.

Deletions of chromosomes can also result in infant disorders. For example, Turner Syndrome is the result of a chromosomal deletion wherein a girl has one X chromosome but an incomplete second X chromosome, oftentimes resulting in infertility and other potential developmental and/or intellectual challenges. A deletion on chromosome 5 can result in Cat Cry Syndrome, resulting in high pitched crying and mental and physical challenges. Prader-Willi Syndrome, a chromosome 15 deletion, can also cause mental and physical obstacles for an individual along with other behavioral issues. A deletion on chromosome 22 resulting in 22q11 Deletion Syndrome can cause various problems such as heart defects, cleft lip/palate issues, immune system disorders and other behavioral and/or physical issues.

Historically, diagnosing chromosomal abnormalities has been done with prenatal testing such as amniocentesis or chorionic villus sampling or after birth using a blood test. However, invasive amniocentesis or chorionic villus sampling can result in premature termination of pregnancy. Further, blood tests serve as a screening mechanism only and are not yet diagnostic; determining the fetal contribution to a maternal blood sample is problematic due in part to the fragility and scarcity of fetal cells that may be circulating in the maternal blood stream or the high degree of experimental variance of different sample types.

As such, what are needed are non-invasive methods and systems that can provide a prenatal diagnosis of fetal chromosomal abnormalities prior to birth. Such methods and systems could provide advance knowledge to diagnosticians, genetic counselors and prospective parents as to the health and wellbeing of an unborn child.

### BRIEF SUMMARY

The present disclosure provides methods and systems for determining the presence of aneuploidy in a sample. In particular, the present disclosure provides noninvasive methods and systems for detecting and diagnosing the presence of aneuploidy in a fetus, mother, father or combinations thereof and for determining the genotype of a fetus.

DNA from maternal blood contains not only that from the mother, but also can contain circulating DNA and/or nucleated red cells from the fetus. As such, if a mixture of DNA fragments from maternal blood is sequenced it can be assumed that sequences read from some of the fragments will originate from fetal DNA. Techniques can be used to exploit this maternal DNA blood profile in attempts to detect fetal aneuploidy (aneuploidy being the duplication or deletion of chromosomes, or a portion of a chromosome), such as trisomy. However, in some cases these techniques can result in excessive noise and false negatives. For example, often in these cases there is a need to establish whether the sample actually contains fetal DNA.

Embodiments set forth herein, such as those using high depth, or deep, sequencing of targeted single nucleotide polymorphisms (SNPs) do not suffer from the same issues. Additionally, by using SNPs one can directly determine the percentage of fetal DNA in the sample and minimize or eliminate false negatives. Embodiments of the sequencing strategy disclosed herein further allow for a fetal genotype to be determined as well as providing a method for paternity testing. Moreover, methods and systems disclosed herein can be used for making a prenatal diagnosis, either alone or in combination with other diagnostic and/or prognostic tests if desired.

As such, the present disclosure provides methods and systems for determining the presence or absence of aneuploidy from the blood of a pregnant female. In embodiments of the present disclosure, blood can be obtained (for example, via blood draw) from a pregnant female. Genomic DNA can be extracted and purified from, for example the serum or plasma fraction of the blood by any means known to those skilled in the art. Following purification of genomic DNA, the DNA can be processed for sequencing. Sequencing provided data can be utilized, either empirically or computationally, for determining the presence or absence of fetal chromosomal anomalies in the sample. Therefore, methods and systems as described herein can be used in determining the presence or absence of a fetal chromosomal anomaly, such as Trisomy 21 or Down's syndrome in a sample. The methods and systems described herein find further utility as a prenatal diagnostic and for determining the genotype of a fetus.

In embodiments of the present application, a blood sample can be obtained from a pregnant female. A maternal blood sample can be separated into two components, a cell containing component and a non-cell component. Genomic DNA can be extracted from the plasma or serum of a non-cell component or from the cellular component. Regardless of source, the genomic DNA extracted from the blood of a pregnant female comprises extraneous fetal DNA, either as circulating DNA (non-cell component) or nuclear DNA as found in circulating fetal nucleated cells (cellular component) such as nucleated fetal red blood cells. Genomic DNA isolated from either fraction may therefore contain a certain percentage of fetal DNA. Although the methods of the present disclosure are exemplified with respect to a mixture of maternal and fetal DNA that is obtained from maternal blood, it will be understood that the methods can be used for mixtures of maternal and fetal nucleic acids (e.g. DNA or RNA) obtained from other bodily fluids, tissues or other biologically derived samples that contain such a mixture. As used herein, nucleic acids can be, for example, a polymer of nucleotides or a polynucleotide. The term can be used to designate a single molecule, or a collection of molecules. Nucleic acids may be double stranded or single stranded and may include coding regions, non-coding regions, regulatory regions, whole chromosomes, partial chromosomes and fragments and variants thereof. Further a blood sample can be derived from any animal for fetal anomaly determination including, but not limited to, humans, non-humans and non-human animals including but not limited to, vertebrates such as rodents, ovines, bovines, ruminants, lagomorphs, porcines, caprines, equines, canines, felines, ayes, etc.

Following genomic DNA extraction and purification, for example from a maternal blood sample, the genomic DNA can be processed for sequencing. Processing may differ depending on which sequencing instrument and technology is being utilized. Once the samples are processed, they can be introduced to the appropriate medium for sequencing on the particular sequencing instrument. Regardless of the sequencing method, sequencing may be performed and nucleotide sequences can be identified. It is desirable that a number of loci are sequenced for practicing the methods and systems disclosed herein, for example at least 20, at least 50, at least 100, at least 200, at least 300, at least 400, at least 500, at least 700, at least 1000, at least 1500 or at least 2000 loci may be sequenced. Two or more alleles can be present at each locus in the sample. Upon sequencing the plurality of loci, allelic frequencies for the reference and non-reference alleles at the loci, ratios of those reference and non-reference alleles and the distribution of the plurality of ratios can be generated to determine fetal aneuploidy.

Different genes can be identified with different loci on a chromosome, wherein each gene, for example, may be associated with one or more different allelic sequences. Alleles are not limited to any specific type and may include, for example, normal genetic sequences or variant genetic sequences. The alleles may be located in loci found on one or more of the chromosomes 1-22, X or Y; however at least one or more loci sequenced is located at the chromosomal location of interest. For example, if determining the presence of trisomy 21 is desired, then a plurality of sequenced loci would be located on chromosome 21. Additionally, it is preferred that loci with a high minor allele frequency (MAF) (i.e., the frequency at which the less common allele occurs in a given population) be chosen for sequencing. For example, in embodiments disclosed herein loci with an MAF of approximately 50% were chosen for sequencing. However, loci with lower MAF's can be utilized in which case more total targeted SNPs should be sequenced. As such, an MAF of at least 3%, at least 5%, at least 10%, at least 20%, at least 30%, at least 40%, at least 60%, at least 70%, at least 80% can also be used in methods and systems as disclosed herein.

In preferred embodiments, deep sequencing is performed on the samples. Deep sequencing can be performed wherein read depth at any particular locus is at least 100×, at least 200×, at least 300×, at least 500×, at least 1000×, at least 2000×, at least 3000×, at least 4000×, at least 5000×, at least 7000× or at least 10,000×. Deep sequencing, in the context of the present disclosure, relates to how many times a particular locus is read or sequenced during the sequencing process. Allele frequencies may be determined for each locus (e.g., wherein the allele frequency reports the number of reads of a particular allele on a background of the total number of reads for that particular locus). The allele frequencies from the alleles sequenced may be compared and aneuploidy determined. A report can be provided containing, among other statistics, a determination of the fetal genotype and a probability of the presence or absence of fetal aneuploidy. In preferred embodiments, alleles sequenced for determining aneuploidy or fetal genotypes comprise single nucleotide polymorphisms.

In some embodiments, the present disclosure provides methods for determining fetal aneuploidy comprising obtaining the sequence of alleles at a plurality of loci in a maternally derived sample comprising maternal and fetal nucleic acids, quantitating a ratio of the reference and non-reference alleles at the plurality of loci, determining the distribution of the ratios of the reference and non-reference alleles at the plurality of loci, and identifying the presence or absence of fetal aneuploidy in said sample based on said distribution of ratios. In some embodiments, a maternal derived sample is a plasma or serum sample. In preferred embodiments, sequencing of the samples is performed by deep sequencing (e.g., using sequence by synthesis methodologies) at a depth of at least 1000×, at least 5000× or at least 10,000× or more. In some embodiments, the reference and non-reference alleles are the same for one or more of the plurality of loci, whereas in other embodiments the reference and non-reference alleles are different for one or more of the plurality of loci. In some embodiments, the non-reference allele is a single nucleotide polymorphism. In preferred embodiments, the loci being queried comprise loci on one or more of chromosomes 8, 9, 13, 18, 21 and/or chromosome 22. In some embodiments, methods disclosed herein identify fetal aneuploidy on Chromosome 21, such as trisomy 21. In some embodiments, quantitating a ratio of the reference and non-reference alleles comprises determining allele frequencies for the reference and non-reference alleles. In some embodiments, determining the distribution of ratios comprises determining the presence or absence of a 0.5:0.5 ratio of reference to non-reference alleles at one or more loci, such that a ratio other than a 0.5:0.5 is indicative of aneuploidy at that locus. In other embodiments, a fetal genotype and/or paternity of the fetus is determined along with the determination of the presence or absence of aneuploidy in a sample. In some embodiments, the percentage of fetal nucleic acids relative to total nucleic acids in a maternal sample for testing is about 5% or less.

In some embodiments, methods are described herein for determining a prenatal diagnosis comprising determining the presence of fetal aneuploidy in a nucleic acid sample derived from a maternal plasma or serum sample according to the methods previously described and providing a diagnosis based on the presence of said fetal aneuploidy. In some embodiments, the prenatal diagnosis determined is trisomy 21, or Down Syndrome.

In some embodiments, a computer implemented method is used for determining the presence or absence of fetal aneuploidy comprising practicing the methods previously described, comprising quantitating the allele frequencies of reference and non-reference alleles at a plurality of loci from a nucleic acid sample comprising fetal nucleic acids, computationally determining the ratio of the allele frequencies of said alleles, generating the distribution of ratios of the alleles, and determining the presence or absence of fetal aneuploidy based on said distribution of ratios. In some embodiments, quantitation comprising sequence the sample in question, for example deep sequencing of at least 1000×, at least 5000× or at least 10000×. In some embodiments, computational analysis is performed on sequence data derived from a maternal sample wherein about 5% or less of the nucleic acids are fetal in origin. In preferred embodiments, the results of the previously described computer implemented methods are output wherein said output could be a diagnosis, for example a diagnosis for trisomy 21. For other embodiments, additional sample related information can be output, such as information with regards to fetal genotype and/or paternity information. Outputting can be by a variety of means as described herein, for example results can be output visually on, for example a computer monitor and the like, or output can be hardcopy, such as a printed paper report and the like.

### DESCRIPTION OF THE DRAWINGS

FIG. 1 exemplifies a simulated sample of 10% normal diploid fetal DNA mixed in maternal DNA. Seven distinct peaks are present. The central distribution contains three peaks representing scenarios when a mother is heterozygous for an allele. The simulated data is based on 2000 SNPs with a MAF=50% frequency in the population and every SNP position is sequenced to a mean depth of 1000×, bin size 0.5%.

FIG. 2 exemplifies a simulated sample demonstrating trisomy. The sample is 10% trisomy fetal DNA in maternal DNA. Six distinct peaks are present; mean sequencing depth of 1000× at 2000 SNPs, bin size 0.5%.

FIG. 3 exemplifies a simulated sample of 10% trisomy fetal DNA in paternal DNA. Ten distinct peaks are present; means sequencing depth of 1000× at 2000 SNPs, bin size 0.5%.

FIG. 4 exemplifies a maternal trisomy as demonstrated in FIG. 2, except with 5% fetal DNA instead of 10%. No distinct peaks are present. The bin size was reduced from 0.5% to 0.1%.

FIG. 5 exemplifies trisomy with maternal duplication as shown in FIG. 4, except the mean sequence depth is increased to 10,000×. Two peaks are distinctly resolved demonstrating that increasing sequencing depth provides increased resolution with small amounts of fetal DNA.

FIG. 6 describes one workflow embodiment of methods and systems disclosed herein. The methods and systems can be used to determine a number of important parameters associated with a sample, including the percentage of fetal DNA in a sample, the presence or absence of fetal aneuploidy and a fetal genotype. A report can be output, for example for use by a diagnostician in prenatal diagnosis.

FIG. 7 provides an algorithm for a statistical determination of the presence or absence of fetal aneuploidy for example trisomy 21, from a maternal blood sample.

FIG. 8 exemplifies a computer hardware communication system embodiment for practicing embodiments as described herein.

### DETAILED DESCRIPTION

DNA derived from the blood of a pregnant woman, in addition to containing the mother's DNA also contains according to some estimates roughly 3-12% circulating fetal DNA. Currently available sequencing methods for detecting fetal trisomy 21 in this mixed sample can be problematic. For example, sequence depth measures may be, excessively noisy leading to ambiguous results with no clear determination or diagnosis possible. Further, with respect to current sequencing methodologies, the power for detecting the differences in the sequence reads may be compromised as the methods depend greatly on the presence of fetal DNA, and yet do not necessarily determine the amount of DNA that is present. This can lead to ambiguities when trying to distinguish a true diagnostic reading from a technical anomaly of the reading itself.

Conversely, the present disclosure describes non-invasive methods and systems for using maternal blood and performing sequencing such as high depth, or deep sequencing, of targeted DNA polymorphisms, such as single nucleotide polymorphisms, methods which are advantageous for decreasing noise and increasing power for determining fetal aneuploidy, such as trisomy 21. Indeed, the methods and systems provided herein are advantageous in detecting trisomy; moreover they can also be utilized to genotype the fetus thereby detecting potentially any chromosomal anomaly which may be present in a fetus. Further, methods and systems described herein can be used for determining the percentage of fetal DNA (relative to total DNA) present in a maternal DNA sample and for determining paternal origin (i.e., paternity testing). An advantage of determining this percentage is that this information can be used to corroborate accuracy of results by ruling out false readings that may have otherwise arisen from technical deficiencies of a sample or problems arising from how the sample was processed. Moreover, the methods and systems described herein can be advantageous to a diagnostician in rendering a prenatal diagnosis to a patient. Further methods and systems described herein can be used to determine whether fetal aneuploidy is maternal or paternal in origin, and for determining paternity of the fetus.

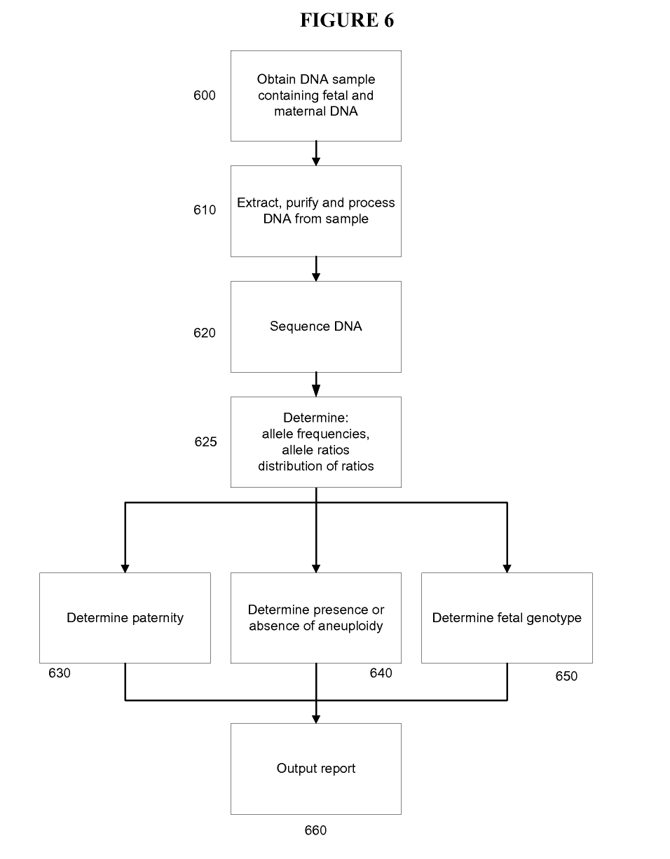

FIG. 6 provides an exemplary embodiment of a method of the present disclosure. A blood sample can be secured from a pregnant female (600) and genomic DNA can be harvested from a fraction of the blood sample (610). The genomic DNA can be sequenced (620) and characteristics, such as allele frequency of a plurality of nucleotide polymorphisms and ratios of the different alleles (i.e., reference and non-reference alleles) and the distribution of the ratios can be determined (625). From the characterizations of the sequenced DNA a number of parameters can be computationally determined. For example, the paternity of the fetus can be determined (630) and/or the presence or absence of one or more additional, non-natural chromosomes (i.e., aneuploidy) in the sample can be determined (640) and/or a fetal genotype can be determined (650), to name only a few. The results can be output in a variety of different ways (e.g., paper, graphic user interface, etc.) (660). The output can then be used by diagnosticians, genetic counselors, parents and the like to determine the status of the fetus.

The term “allele” is used consistent with its meaning in the art of biology. An allele can be one or more alternative forms of a gene or genetic sequence found at a specific location, or locus, on a chromosome. As used herein, a “reference” allele is one form of a gene or genetic sequence and a “non-reference” allele comprises the same or alternative forms of that reference allele. A reference allele can be inherited from the mother (i.e., maternally derived allele), whereas a non-reference allele can be an allele inherited from the father (i.e., paternally derived allele), or vice versa. For example, a reference allele could be a nucleotide (e.g., adenine) as inherited from mother and the non-reference allele could be any of the other three nucleotides (e.g., thymine, cytosine and guanine) or the sample nucleotide (i.e., adenine) as inherited from the father, or vice versa. In another example, a reference allele could be a wild type or normal allele (i.e., a nucleotide as observed in a Human reference sequence database for example, UCSC Genome Browser, HapMap database, etc.) as inherited from the mother, whereas a non-reference allele could by a single nucleotide polymorphism (SNP) or another variant of the normal or wild type allele as inherited from the father, or vice versa. As such, it is not required that all of the alleles being sequenced be associated with known polymorphic sequences. For example, for practicing the methods disclosed herein one or more of the alleles being sequenced may have known polymorphisms associated with that allele. Conversely, it may not be known whether one or more of the alleles is polymorphic. As such, it is not necessary that the maternal and/or paternal genotype be known prior to sequencing. Any allele can be utilized in methods disclosed herein regardless of whether or not they are associated with known polymorphisms.

The term “locus” is used consistent with its meaning in the art of biology. A locus (plural “loci”) refers to a specific location or place on a chromosome identified with a gene or genetic sequence, such as an allele, SNP, etc.

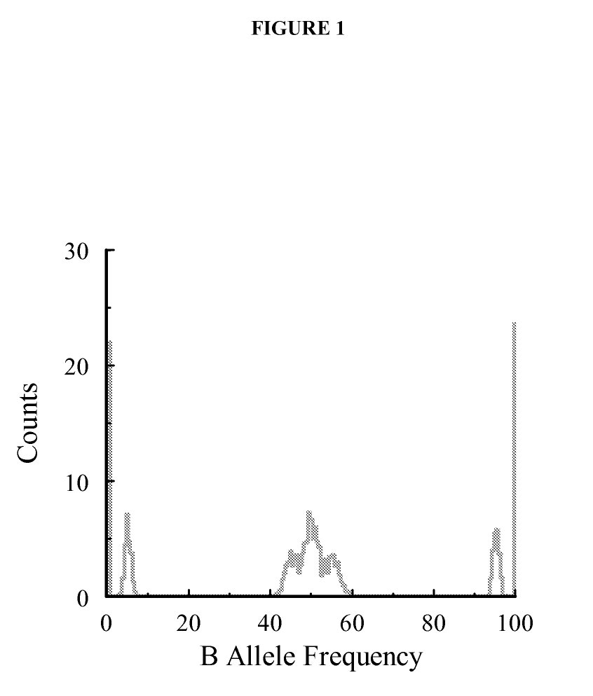

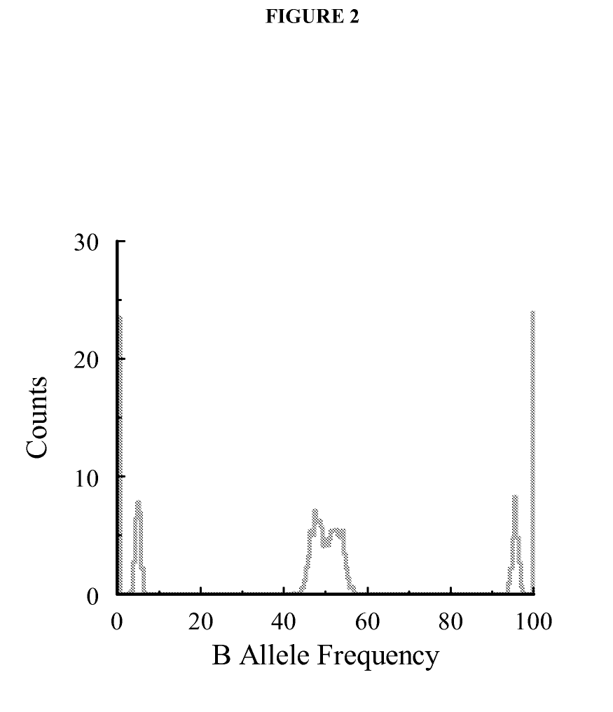

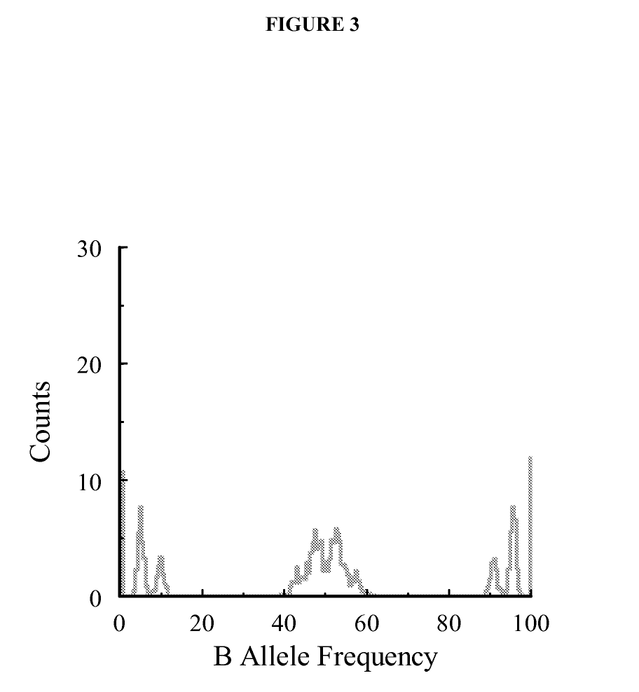

The term “distribution of ratios” as used herein describes the composite of the ratios calculated from the reference and non-reference allelic sequence data generated from the loci chosen for sequencing. As such, “distribution of ratios” corresponds to the measurements of the ratios at many different polymorphic positions queried and their distribution. For example, an investigator may choose to sequence a panel of 1000 loci to determine the presence or absence of aneuploidy. Once the sequence of the reference and non-reference alleles is determined a ratio of reference to non-reference alleles can be generated for the loci. In this example, the 1000 ratios would be evaluated together. Continuing with the example, a distribution consistent with the peaks represented in exemplary FIG. 1 that includes a peak with a ratio of 0.5:0.5 (i.e., one of the two alleles from the mother and the other allele from the father) would demonstrate that at that particular location there is no fetal trisomy. However, if the distribution is consistent with multiple peaks excluding a peak with a ratio of 0.5:0.5, then that difference would be indicative of aneuploidy (for example, FIGS. 2-3). As such, by evaluating the distribution of a plurality of ratios (e.g. from all the queried alleles at the plurality of loci, in this example 1000 loci) the presence or absence of aneuploidy can be determined for any chromosome or target chromosomal region. The details of embodiments are set forth in the accompanying drawings and the descriptions below. Other features, objects, and advantages will be apparent from the description and drawings, and from the claims.

According to some non-limiting estimates, approximately 3-12% of the DNA found in maternal blood can be found as fetal DNA. The methods set forth herein are useful for samples having fetal DNA percentages inside this range and even outside of this range. Sequencing data obtained from maternal blood can therefore contain sequence information, or sequence reads, derived from both maternal chromosomes and, through the fetal DNA, from any inherited one of the paternal chromosomes. For example, assuming normal diploid parental genomes, if the fetal DNA represents some fraction (X) of the total DNA then X/2 of the total number of sequence reads is contemplated to be derived from the paternal DNA. As such, when a fraction X of the total DNA is derived from fetal DNA, the following proportions of maternal and paternal DNA are contemplated to be present in a blood sample from a pregnant woman:

f₁=x/2; f₂=0; m₁=½; m₂=(1−x)/2 (1)

wherein f₁ and f₂ represent the two paternal chromosomes, m₁ and m₂ represent the two maternal chromosomes, wherein m₁ and f₁ are the maternal and paternal chromosomes inherited by the fetus and wherein m₂ and f₂ are the maternal and paternal chromosomes not present in the fetus.

For example, the detection of biallelic markers such as single nucleotide polymorphism loci (SNPs) where there is the possibility of two alleles, for example a reference allele and a non-reference allele (inherited from mother and father, or vice versa), can lead to a distinctive fingerprint in the non-reference-allele frequencies wherein the non-reference allele frequency is the fraction/percentage of overall sequence reads that identify one of the two alleles. In demonstration, given two possible alleles (arbitrarily called allele A (reference) and allele B (non-reference)) at a particular chromosomal location the expected B-allele frequencies in the blood-derived maternal DNA sample would be:

0, x/2, (1−x)/2, ½, (1+x)/2, 1−x/2, 1 (2)

wherein the first and last terms (0 and 1, respectively) represent a DNA sample where both the fetal and maternal DNA are homozygous for the same allele and wherein the second and second to last terms (x/2 and 1−x/2, respectively) represent the case where the fetus is heterozygous for the allele and the maternal DNA is homozygous for the allele. At a given locus on a chromosome both a reference and non-reference allele (alleles A and B, respectively) are possible, for example allele A representing the observed base from the Human reference sequence and allele B representing one of three possible non-reference bases. At each known polymorphic site, non-reference allele frequencies (e.g. a proportion or percentage) can be determined from the total ratio of reference and non-reference alleles that are observed in a sample and the distribution of those ratios.

It is contemplated that if a sufficient number of SNPs are measured at a sufficient depth a plot of the binned counts of non-reference allele frequency can be graphed. FIG. 1 exemplifies a simulated DNA plot based on SNPs that occur with at least 50% minor allele frequency (MAF) in a simulated population wherein 10% of the DNA sequenced is derived from the fetus. Sequencing the sample at 1000× for each of 2000 SNPs, peaks at 0, 0.05, 0.45, 0.50, 0.55, 0.95 and 1 are demonstrated. As previously described, the two peaks at x/2 (0.05) and 1−x/2 (0.95) represent positions wherein the paternal allele is different from both maternal alleles and can be used to detect the paternal alleles passed to the fetus.

In one embodiment paternity testing can also be performed utilizing the methods and systems herein described. For example, if only 100 SNPs from the x/2 and 1−x/2 peaks were sequenced, assuming that all SNPs occur at 50% frequency in the population, it could be expected that the true father would have all 100 SNPs in his genome whereas a randomly selected potential father would only be expected to carry 75 of the SNPs (p<10⁻⁶). As such, paternity could be determined utilizing methods as described herein.

It is contemplated that for any chromosomal abnormality that results in an odd number of fetal chromosomes sampled (assuming that the mother is diploid) the 50% fraction peak depicted in FIG. 1 would not exist. Maternal trisomy, the case wherein the fetus carries two chromosomes inherited from the maternal DNA (m₁ and m₂) and one chromosome from the paternal DNA (f₁), is the most common form of trisomy. For maternal trisomy wherein the fetus contains one copy each of f₁, m₁ and m₂ the fraction of paternal and maternal chromosomes in the mother's blood can be demonstrated as:

f₁=x/3; f₂=0; m₁=m₂=½−x/6 (3)

In the maternal trisomy example, B-allele frequency peaks are expected to occur at:

0, x/3, ½−x/6, ½+x/6, 1−x/3, 1 (4)

Given a sufficient number of SNPs, this example would produce two distinct peaks to either side of the 0.5 (50%) MAF mark without a peak at 0.5, indicative of fetal trisomy. This trisomy pattern is exemplified in FIG. 2. The peaks around 0.5 are contemplated to become more distinct, for example as the fraction of fetal DNA increases in a sample and/or the SNP read depth increases. Conversely, the peaks could also merge towards one another with lower amounts of fetal DNA and/or lower read depths.

While maternal trisomy is the most common cause of Trisomy 21, paternal trisomy, where the fetal DNA carries two chromosomes from the paternal DNA, can also occur. For paternal trisomy, wherein the fetus contains one copy each of f₁, f₂ and m₁, the fraction of paternal and maternal chromosomes in the mother's blood would be:

f₁=f₂=x/3; m₁=(1−x)/2+x/3; m₂=(1−x)/2 (5)

In the paternal trisomy example, ten B-allele frequency peaks are demonstrated in FIG. 3 as expected and occur at:

0, x/3, 2*x/3, 0.5−x/6, 0.5−x/2, 0.5+x/2, 0.5+x/6, 1−2*x/3, 1−x/3, 1 (6)

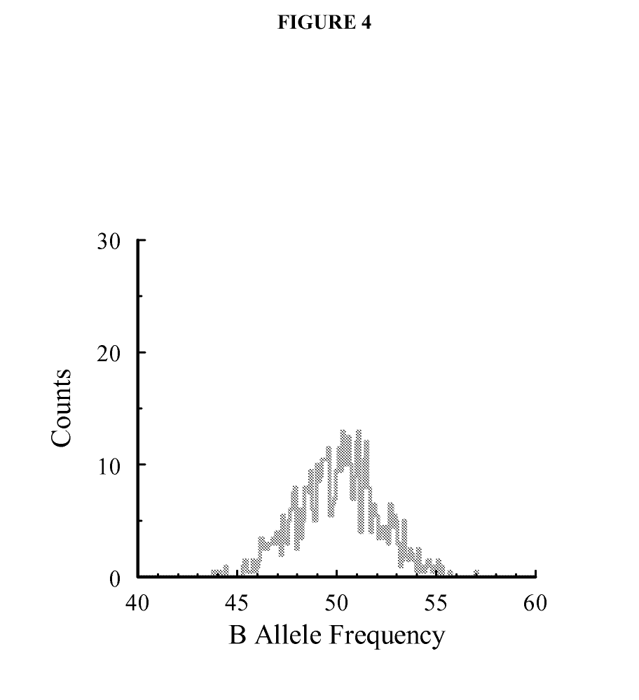

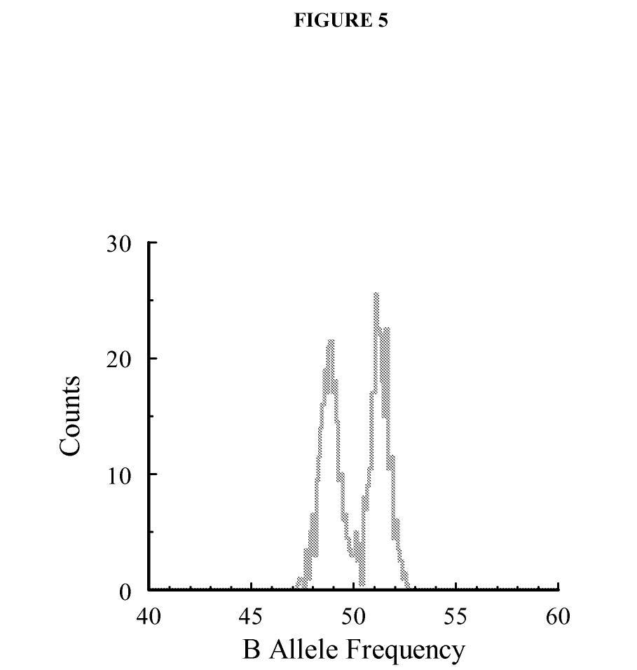

As the percentage of fetal DNA decreases in a sample, the ability to visually detect the trisomy also becomes lower. For example, as the percent fetal DNA drops to 5% the two peaks around 50% seen in FIG. 2 become merged into a single peak as demonstrated in FIG. 4. When the percentage of fetal DNA drops in a sample, it can be difficult to determine a positive or negative result. Wherein algorithmic models can be created to resolve the most likely scenario mathematically, it is contemplated that determining a genotype when the fetal DNA drops in a sample can be advantageously resolved by increasing the sequencing depth of the SNPs which will in turn decrease the variance in the two (or three) peaks. Indeed, as exemplified in FIG. 5 increasing the mean sequencing depth to 10,000× clearly resolves the two peaks and provides for a determination of genotype.

As exemplified in the Figures and as described in examples in the present disclosure, evidence for fetal chromosomal anomalies, such as trisomy, can be determined empirically by graphing the distribution of allelic ratios (reference and non-reference alleles at a multitude of SNPs) such as that demonstrated in FIGS. 1-5. However, chromosomal anomalies be also be determined computationally by computer implemented methods that can distinguish, for example, FIG. 1 wherein the fetal DNA is diploid from FIG. 4 wherein the fetal DNA is triploid but wherein the fraction of fetal DNA in the maternal DNA population is decreased such that distinct peaks are not discernible. As such, the present disclosure provides methods and systems for empirical and computational sequence analysis for determining the presence of chromosomal aneuploidy in a maternal sample.

For example, the number of an A allele in a sample is distributed as a mixture of binomial random variables wherein each component (i.e., chromosomal) mixture has the probability mass function of:

$$( \begin{matrix}n \\ k\end{matrix} ) {{μ}^{k} ( 1 - μ )}^{n - k} \qquad ( 7 )$$

wherein n is the number of observed A alleles plus the number of observed B alleles, k is the number of observed A alleles, and μ is the rate at which the A allele is expected to occur.

The variables n and k are provided by the sequencing experiment wherein duplicate reads have been removed. The number of components or chromosomes in each mixture, and the μ for each component, correspond to the location of peaks described in equations (2), (4) and (6). It is contemplated that the values may be slightly different from the simulation values described earlier, for example due to substitution errors present in the sequencing-by-synthesis platform which can occur from platform to platform. However, the error rates are anticipated to be below 1% and can be modeled with small adjustments as known to a skilled artisan, as such not detracting from the genotyping or determination of chromosomal abnormalities.

To fully describe a binomial mixture model for each of three models previously described (i.e., normal diploid fetal DNA, maternal trisomy and paternal trisomy) and for each SNP (i.e., normal diploid fetal DNA, maternal trisomy, and paternal trisomy) mixing proportions can be utilized. The mixing proportions can be derived using known allele frequencies at each marker (i.e., SNP) in the population being considered. The mixing proportions for each of the three models is as follow, wherein p is the frequency of the B allele in the population and q=1−p is the frequency of the A allele:

Normal Pregnancy: q³, q²p, q²p, qp, qp², qp², p³
Maternal Trisomy: q³, q²p, 2q²p, 2qp², qp², p³
Paternal Trisom q⁴, 2q³p, q²p², q³p, 2q²p²+q³p, 2q²p²+qp³, qp³, q²p², 2qp³, p⁴

Given a genetic model (i.e., normal diploid fetal DNA, maternal trisomy or paternal trisomy) a likelihood of a sample reporting a fetal chromosomal abnormality can be calculated when 1) a percentage of reads of fetal origin, 2) an error rate, 3) observed counts of the A and B allele, and 4) expected frequencies of these alleles in the wider population are determined. Using Bayes' Theorem, one can turn the data into a likelihood of the model by, for example, multiplying the likelihoods across all SNPs for a given model and then multiplying this product by the prior probability of the model. In reality, these “priors” will have minimal effect when many SNPs are sequenced to a high depth, for example at least 50 SNPs, at least 100 SNPs, at least 150 SNPs, at least 200 SNPs, at least 300 SNPs, at least 500 SNPs, at least 1000 SNPs, at least 2000 SNPs, etc. In preferred embodiments, at least 100 SNPs are utilized to determine ratios of reference and non-reference alleles and the distribution of ratios for determining fetal aneuploidy. However, a flat prior, where all models are treated equally, could be utilized as could rates that have been previously reported.

The likelihood of each model over this unknown parameter (i.e., unknown fetal DNA contribution to a maternal blood sample) is maximized in the disclosed methods. For example, the combination of percent of fetal DNA and the model that yields the highest likelihood is the model chosen for the data. Optionally, one could also maximize over the error rate, however the q-score data would provide a good estimate of the error rate.

As a control, a fourth model that corresponds to a “not pregnant” sample could be evaluated for comparison to the maternal test sample. Indeed, including such a control in an assay would be advantageous in identifying nonsense results, for example if a male blood sample were inadvertently tested. Further, additional negative and positive controls could be added that correspond to artificial SNPs. The artificial SNPs could be added, or spiked into, a sequencing reaction to mimic the various models being assayed and evaluated.

It is contemplated that the methods and systems described herein would find utility in determining a fetal chromosomal anomaly, in determining a genotype of a fetus and in providing a prenatal diagnosis. Such determination could be used to identify the presence of, and diagnosing of, fetal chromosomal abnormalities, such as Down syndrome (trisomy 21) Patau syndrome (trisomy 13), Edwards syndrome (trisomy 18), Trisomy 9, Trisomy 16, Warkany syndrome 2 (trisomy 8) and Cat eye syndrome (trisomy 22), triploidy, polyploidy, tetraploidy, segmental aneuploidy such as duplications in areas of a chromosome (e.g., 1p36, 17p11.2, 22q11.2), Cri du chat syndrome, to name but a few. As such, as described herein non-invasive methods (e.g., blood draw) can be used to generate data for comparing a plurality of genetic polymorphisms, such as SNPs, at different locations on one or more chromosomes for determining aneuploidy and/or genotype of a fetus.

It is further contemplated that the methods and systems described herein would find utility in paternity testing as the identified SNPs used for determining the presence of aneuploidy can be used to determine which contribution is not only maternal, but also which contribution is paternal. Methods and systems described herein further provide for determining the percentage of fetal DNA present in a maternal sample.

A number of embodiments have been described. Nevertheless, it will be understood that various modifications may be made. Accordingly, other embodiments are within the scope of the present application.

In embodiments of the present disclosure, a blood sample can be procured from a pregnant female. The blood sample can be separated into components, one component containing cells and other containing either plasma or serum, depending on whether an anticoagulant was in contact or not, respectively, with the blood. Genomic DNA can be extracted from the cell free component (i.e., plasma or serum). Embodiments as described herein are not limited by the nucleic acid preparatory methods and any number of methods may be practiced by a skilled artisan in order to provide nucleic acids for use in disclosed methods. For example, any number of commercially available genomic DNA extraction and purification kits such as, for example, the MasterPure™ Complete DNA and RNA purification kit (Epicentre®, Madison, Wis.), QIAamp DNA Mini Kit (Qiagen, Valencia, Calif.) or ReliaPrep™ Blood gDNA miniprep system (Promega, Corp., Madison, Wis.) or following any number of available protocols such as described in Molecular Cloning: A Laboratory Manual (Eds., Sambrook, Fritsch and Maniatus, Cold Spring Harbor Laboratory) can be used to extract genomic DNA from a sample.

Following genomic DNA extraction from the maternal sample, the genomic DNA can be processed for sequencing. Processing may differ depending on which sequencing instrument and technology is being utilized. Methods and systems disclosed herein are not limited to any particular processing and sequencing methods. For example, fragment libraries can be created from isolated genomic DNA for sequencing. A library is produced, for example, by performing the methods as described in the Nextera™ DNA Sample Prep Kit (Epicentre® Biotechnologies, Madison Wis.), SOLiD™ Library Preparation Kits (Applied Biosystems™ Life Technologies, Carlsbad Calif.), and the like. A DNA library sample may be further amplified for sequencing by, for example, multiple stand displacement amplification (MDA) techniques.

For sequencing, a sample library is, for example, prepared by creating a DNA library as described in Mate Pair Library Prep kit, Genomic DNA Sample Prep kits or TruSeq™ Sample Preparation and Exome Enrichment kits (Illumina®, Inc., San Diego Calif.). Alternatively, a library may be prepared without amplification. For example, a sample can be sheared or digested to yield fragments of different lengths and the desired fragment size can be selected (e.g., gel electrophoresis, size selection column, etc.). The fragments can be modified to comprise at one or more of their 5′ and 3′ ends a selection sequence for example adapter sequences that are added (for example, by ligation) to one or more end of the fragment, poly-A tailing, and the like. These types of non-amplified libraries are used, for example, for single molecule sequencing technologies such as those practiced in the HeliScope™ Single Molecule Sequencer (Helicos BioScience Corporation, Cambridge, Mass.). A skilled artisan will recognize additional methods and technologies for preparing nucleic acid libraries which could also be used in combination with the methods and compositions described herein. Embodiments described herein are not limited to any amplification or non-amplification library preparation method.

DNA libraries can be immobilized on a substrate, such as a flowcell, and bridge amplification performed on the immobilized polynucleotides prior to sequencing, for example sequence by synthesis methodologies. In bridge amplification, an immobilized polynucleotide (e.g., from a DNA library) is hybridized to an immobilized oligonucleotide primer. The 3′ end of the immobilized polynucleotide molecule provides the template for a polymerase-catalyzed, template-directed elongation reaction (e.g., primer extension) extending from the immobilized oligonucleotide primer. The resulting double-stranded product “bridges” the two primers and both strands are covalently attached to the support. In the next cycle, following denaturation that yields a pair of single strands (the immobilized template and the extended-primer product) immobilized to the solid support, both immobilized strands can serve as templates for new primer extension. Thus, the first and second portions can be amplified to produce a plurality of clusters. The terms “cluster” and “colony” are used interchangeably and refer to a plurality of copies of a nucleic acid sequence and/or complements thereof attached to a surface. Typically, the cluster comprises a plurality of copies of a nucleic acid sequence and/or complements thereof, attached via their 5′ termini to the surface. Exemplary bridge amplification and clustering methodology are described, for example, in PCT Patent Publ. Nos. WO00/18957 and WO98/44151, U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2005/0100900, U.S. Patent Publ. No. 2004/0002090; U.S. Patent Publ. No. 2007/0128624; and U.S. Patent Publ. No. 2008/0009420, each of which is incorporated herein by reference in its entirety. The compositions and methods as described herein are particularly useful in sequence by synthesis methodologies utilizing a flowcell comprising clusters.

Emulsion PCR methods for amplifying nucleic acids prior to sequencing can also be used in combination with methods and systems as described herein. Emulsion PCR comprises PCR amplification of an adaptor flanked shotgun DNA library in a water-in-oil emulsion. The PCR is multi-template PCR; only a single primer pair is used. One of the PCR primers is tethered to the surface (5′ attached) of microscale beads. A low template concentration results in most bead-containing emulsion microvesicles having no more than one template molecule present. In productive emulsion microvesicles (an emulsion microvesicle where both a bead and template molecule are present), PCR amplicons can be captured to the surface of the bead. After breaking the emulsion, beads bearing amplification products can be selectively enriched. Each clonally amplified bead will bear on its surface PCR products corresponding to amplification of a single molecule from the template library. Various embodiments of emulsion PCR methods are set for in Dressman et al., Proc. Natl. Acad. Sci. USA 100:8817-8822 (2003), PCT Patent Publ. No. WO 05/010145, U.S. Patent Publ. Nos. 2005/0130173, 2005/0064460, and US2005/0042648, each of which is incorporated herein by reference in its entirety.

DNA nanoballs can also be used in combination with methods and systems as described herein. Methods for creating and utilizing DNA nanoballs for genomic sequencing can be found at, for example, US patents and publications U.S. Pat. No. and publications 7,910,354, 2009/0264299, 2009/0011943, 2009/0005252, 2009/0155781, 2009/0118488 and as described in, for example, Drmanac et al., 2010, Science 327(5961): 78-81; all of which are incorporated herein by reference in their entireties. Briefly, following genomic DNA fragmentation consecutive rounds of adaptor ligation, amplification and digestion results in head to tail concatamers of multiple copies of the circular genomic DNA template/adaptor sequences which are circularized into single stranded DNA (e.g. by ligation with a circle ligase) and rolling circle amplified (for example, as described in Lizardi et al., Nat. Genet. 19:225-232 (1998) and US 2007/0099208 A1, each of which is incorporated herein by reference in its entirety). The adaptor structure of the concatamers promotes coiling of the single stranded DNA thereby creating compact DNA nanoballs. The DNA nanoballs can be captured on substrates, preferably to create an ordered or patterned array such that distance between each nanoball is maintained thereby allowing sequencing of the separate DNA nanoballs.

Disclosed methods for determining aneuploidy and/or fetal genotype find particular utility when used in sequencing, for example sequencing by synthesis (SBS) technologies. Sequencing by synthesis generally comprises sequential addition of one or more labeled nucleotides to a growing polynucleotide chain in the 5′ to 3′ direction using a polymerase. The extended polynucleotide chain is complementary to the nucleic acid template, which can be affixed on a substrate (e.g., flowcell, chip, slide, etc.), and which contains the target sequence. The labeled nucleotides that are used in SBS can include any of a variety of fluorophores, mass labels, electronically detectable labels or other types of labels. The labeled nucleotides that are used in SBS can also include reversible terminator groups such that only one nucleotide is added per SBS cycle. After the incorporated nucleotide is detected a deblocking agent can be added to render the added nucleotide competent for extension in a subsequent cycle. SBS methods are particularly useful for parallel analysis of different-sequence fragments of a nucleic acid sample. For example, hundreds, thousand, millions or more different-sequence fragments can be sequenced simultaneously on a single substrate using known SBS techniques. Exemplary sequencing methods are described, for example, in Bentley et al., Nature 456:53-59 (2008), WO 04/018497; U.S. Pat. No. 7,057,026; WO 91/06678; WO 07/123,744; U.S. Pat. No. 7,329,492; U.S. Pat. No. 7,211,414; U.S. Pat. No. 7,315,019; U.S. Pat. No. 7,405,281, and US 2008/0108082, each of which is incorporated herein by reference.

Disclosed methods for determining fetal genotype also find utility when used in sequencing by ligation, sequencing by hybridization, and other sequencing technologies where deep sequencing can be performed. An exemplary sequence by ligation methodology is di-base encoding (e.g., color space sequencing) utilized by Applied Biosystems' SOLiD™ sequencing system (Voelkerding et al., 2009, Clin Chem 55:641-658; incorporated herein by reference in its entirety).

The methods for fetal genotyping disclosed herein could be utilized in sequence by hybridization technologies. Sequence by hybridization comprises the use of an array of short sequences of nucleotide probes to which is added fragmented, labeled target DNA (for example, as described in Drmanac et al., 2002, Adv Biochem Eng Biotechnol 77:75-101; Lizardi et al., 2008, Nat Biotech 26:649-650, U.S. Pat. No. 7,071,324; incorporated herein by reference in their entireties). Further improvements to sequence by hybridization can be found at, for example, US patent application publications 2007/0178516, 2010/0063264 and 2006/0287833 (incorporated herein by reference in their entireties). Sequencing approaches which combine hybridization and ligation biochemistries have been developed and commercialized, such as the genomic sequencing technology practiced by Complete Genomics, Mountain View, Calif.). For example, combinatorial probe-anchor ligation, or cPAL™ (Drmanac et al., 2010, Science 327(5961): 78-81) utilizes ligation biochemistry while exploiting advantages of sequence by hybridization. The methods for fetal genotyping disclosed herein could be utilized in combinatorial probe-anchor ligation sequencing technologies.

Single molecule sequencing can also be used with methods as disclosed herein. For example, non-amplified DNA libraries for sequencing can be prepared as previously described. The library fragments can be hybridized and captured on a substrate such as a flow cell and assayed on, for example, a HeliScope™ Single Molecule Sequence instrument. Further description of single molecule sequencing can be found at, for example, Puchkarev et al. (2009, Nat. Biotechnol. 27:847-52, incorporated herein by reference in its entirety) and Thompson and Steinmann (2010, Curr. Prot. Mol. Biol. Cpt 7, Unit 7.10, incorporated herein by reference in its entirety).

Methods as described herein are not limited by any particular sequencing sample preparation method and alternatives will be readily apparent to a skilled artisan and are considered within the scope of the present disclosure. However, particular utility is found when applying the methods herein to sequencing devices such as flow cells or arrays for practicing sequence by synthesis methodologies or other related sequencing technologies such as those practiced by one or more of polony sequencing technology (Dover Systems), sequencing by hybridization fluorescent platforms (Complete Genomics), sTOP technology (Industrial Technology Research Institute) and sequencing by synthesis (Illumina, Life Technologies).

In some embodiments, the methods set forth herein can be used in sequencing system such as those provided by Illumina®, Inc. (HiSeq 1000, HiSeq 2000, Genome Analyzers, MiSeq, HiScan systems), Applied Biosystems™ Life Technologies (ABI PRISM® Sequence detection systems, SOLiD™ System, Ion PGM™ Sequencer, Ion Proton™ Sequencer), Oxford Nanopore Technologies® (GridION, MinION) or other sequencing instrument, further as those described in, for example, United States patents and U.S. Pat. Nos. 5,888,737, 6,175,002, 5,695,934, 6,140,489, 5,863,722, 2007/007991, 2009/0247414, 2010/0111768 and PCT application WO2007/123744, and U.S. patent application Ser. Nos. 61/431,425, 61/431,440, 61/431,439, 61/431,429, 61/438,486 each of which is incorporated herein by reference in its entirety.

Output from a sequencing instrument can be of any sort. For example, several commercial embodiments utilize a light generating readable output, such as fluorescence or luminescence. However the present methods are not limited to the type of readable output as long as differences in output signal for a particular sequence of interest is potentially determinable.

Examples of analysis software that may be used to characterize output derived from practicing methods as described herein include, but are not limited to, Pipeline, CASAVA and GenomeStudio data analysis software (Illumina®, Inc.), SOLiD™, DNASTAR® SeqMan® NGen® and Partek® Genomics Suite™ data analysis software (Life Technologies), Feature Extraction and Agilent Genomics Workbench data analysis software (Agilent Technologies), Genotyping Console™, Chromosome Analysis Suite data analysis software (Affymetrix®).

In methods and systems described herein, one or more data analysis programs comprise computer implemented methods for determining allele frequencies, ratios of the alleles, and distribution of ratios for determining aneuploidy by computational methods. For example, with regards to FIG. 1 a sample from a pregnant female can be processed and sequenced. During sequencing, a computer implemented method could identify, for example, 2000 single nucleotide polymorphism loci (SNPs) distributed among one or more chromosomes, wherein at least some, if not all, of the SNPs are located on chromosome 21. The method can implement algorithms comprising principles and computations of Equations 1-4, such as for determining allele frequencies, allelic ratios and/or the distribution of ratios and comparing the results of the computations to determine whether aneuploidy is present in the sample. The computer implemented methods could also determine, as explained in the principles and computations of Equations 1-6, whether the aneuploidy was maternal or paternal in origin. The computer implemented method could execute the outputting of a report that could be used to provide a prenatal diagnosis of the presence or absence of fetal aneuploidy, for example a diagnosis of trisomy 21 or Down syndrome in a fetus. A report may provide, among other statistics, ratios of reference and non-reference alleles and their distribution, allelic frequencies, a determination of the presence or absence of aneuploidy, a maternal and/or fetal genotype and/or a paternal genotype. In some embodiments, the outputted report will provide information for a diagnostician to render a prenatal diagnosis.

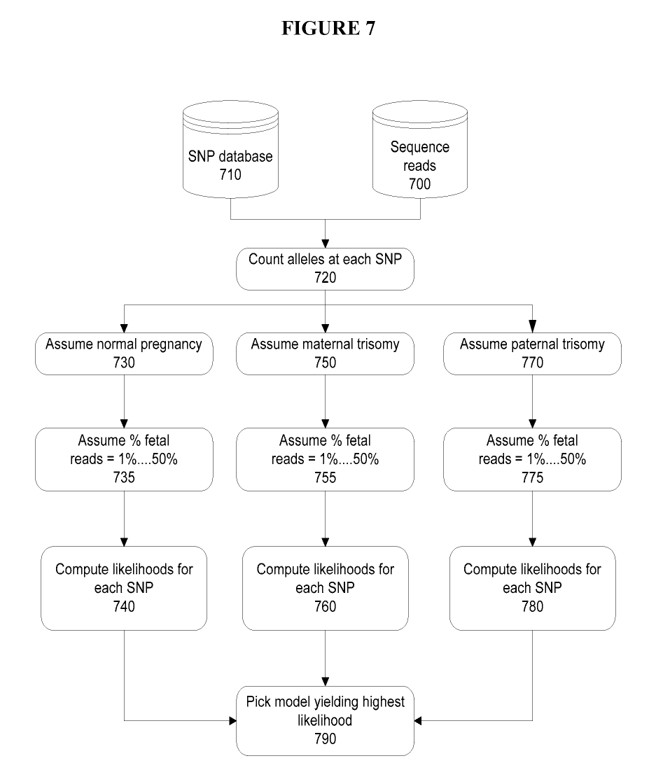

In some embodiments, methods provided herein comprise computer implemented methods and systems for determining aneuploidy and/or a fetal genotype. In some embodiments, a fetal chromosomal anomaly determinable by the computer implemented methods described herein comprises anomalies associated with an increase in the number of chromosomes such as those found in polysomy or trisomy as compared to a normal chromosomal complement. In some embodiments, the computer implemented methods determine the presence or absence of trisomy, or Down syndrome, in a sample. FIG. 7 is an exemplary flowchart of a computer implemented method embodiment of the present disclosure. Sequence reads can be generated upon sequencing a sample (700) and loci of interest can be looked up in a SNP database (710). The alleles at each of the loci can be counted (720). Three potential outcome model assumptions can be investigated; fetal chromosomal complement is normal (730), maternal trisomy (750) or paternal trisomy (770). For each of these three models, it is further assumed that each of 1, 2, 3, 4, 5, . . . −50% of the sample total reads (735, 755 and 775) are of fetal origin. For each of these models (730, 750, 770) the likelihood of the observed allele ratios can be calculated (740, 760, 780). The outcome model that comprises the highest computed likelihoods is chosen (790); either the sample is normal (i.e., no aneuploidy) if the normal pregnancy outcome model assumption yields the highest likelihood, or the sample demonstrates the presence of aneuploidy if the outcome model assumptions for either maternal or paternal trisomy gives the highest likelihood.

Advantages of practicing the computer implemented methods and systems as described herein can provide clinicians, diagnosticians, genetic counselors, etc. with a diagnostic tool for determining the chromosomal complement, and anomalies thereof, of a fetus. Further, information gained by practicing computer implemented methods and systems as described herein finds utility in personalized healthcare initiatives wherein an individual's genomic sequence may provide a clinician with information unique to a patient for diagnosis and specialized treatment prior to birth.

In some disclosed embodiments, methods and systems are provided for computational analysis of large data sets generated by sequencing a genome. Accordingly, disclosed embodiments may take the form of one or more of data analysis systems, data analysis methods, data analyses software and combinations thereof. In some embodiments, software written to perform methods as described herein is stored in some form of computer readable medium, such as memory, CD-ROM, DVD-ROM, memory stick, flash drive, hard drive, SSD hard drive, server, mainframe storage system and the like. A skilled artisan will understand the basics of computer systems, networks and the like, additional information can be found at, for example, Introduction to Computing Systems (Pat and Patel, Eds., 2000, 1^{st} Ed., McGraw Hill Text), Introduction to Client/Server Systems (1996, Renaud, 2^{nd} Edition, John Wiley & Sons), both of which are incorporated herein by reference in their entireties.

Computer software products comprising computer implemented methods for determining aneuploidy and/or a fetal genotype as described herein may be written in any of various suitable programming languages, for example compiled languages such as C, C#, C++, FORTRAN, and Java. Other programming languages could be script languages, such as Perl, MatLab, SAS, SPSS, Python, Ruby, Pascal, Delphi, R and PHP. In some embodiments, the computer implemented methods comprising the principles and algorithms as described herein are written in C, C#, C++, Fortran, Java, Perl, R, Java or Python. In some embodiments, the computer software product may be an independent application with data input and data display modules. Alternatively, a computer software product may include classes wherein distributed objects comprise applications including computational methods as described herein. Further, computer software products may be part of a component software product for determining sequence data, including, but not limited to, computer implemented software products associated with sequencing systems offered by Illumina, Inc. (San Diego, Calif.), Applied Biosystems and Ion Torrent (Life Technologies; Carlsbad, Calif.), Roche 454 Life Sciences (Branford, Conn.), Roche NimbleGen (Madison, Wis.), Cracker Bio (Chulung, Hsinchu, Taiwan), Complete Genomics (Mountain View, Calif.), GE Global Research (Niskayuna, N.Y.), Halcyon Molecular (Redwood City, Calif.), Helicos Biosciences (Cambridge, Mass.), Intelligent Bio-Systems (Waltham. MA), NABsys (Providence, R.I.), Oxford Nanopore (Oxford, UK), Pacific Biosciences (Menlo Park, Calif.), and other sequencing software related products for determining sequence from a nucleic acid sample.

In some embodiments, computer implemented methods for determining aneuploidy and/or fetal genotype as described herein may be incorporated into pre-existing data analysis software, such as that found on or in communication with, sequencing instruments. An example of such software is the CASAVA Software program (Illumina, Inc.; see CASAVA Software User Guide as an example of the program capacity, incorporated herein by reference in its entirety). Software comprising computer implemented methods as described herein are installed either onto a computer system directly, or are indirectly held on a computer readable medium and loaded as needed onto a computer system. Further, software comprising computer implemented methods described herein can be located on computers that are remote to where the data is being produced, such as software found on servers and the like that are maintained in another location relative to where the data is being produced, such as that provided by a third party service provider.

Output of practicing the computational methods as described can be via a graphic user interface, for example a computer monitor or other display screen. In some embodiments, output is in the form of a graphical representation, a web based browser, an image generating device and the like. In some embodiments, a graphical representation is available to a user at any point in time during sequencing data acquisition, for example after one cycle, five cycles, 10 cycles, 20 cycles, 30 cycles or more, thereby providing a user a graphical representation of the sequence of interest as the sequencing reaction progresses. However, in some embodiments output can be in the form of a flat text file that contains sequence information, wherein the text file is added to for each subsequent cycle thereby providing a text file reporting of the sequence of interest as the sequencing reaction progresses. In other embodiments, output is a graphical and/or flat text file that is assessed at the end of a sequencing analysis instead of at any point during a sequencing analysis. In some embodiments, the output is accessed by a user at any point in time during or after a sequencing run, as it is contemplated that the point during a reaction at which the output is accessed by the user does not limit the use of the computational methods. In some embodiments, output is in the form of a graph, picture, image or further a data file that is printed, viewed, and/or stored on a computer readable storage medium. In some embodiments, a graphic representation can be provided as a line graph, for example like the exemplary line graphs of FIGS. 1-5. In some embodiments, the output is provided as a report which comprises a sample characterization which may include, but is not limited to, a line graph (as exemplified in FIGS. 1-5), reference and non-reference allele frequencies, ratios of reference and non-reference alleles, distribution of ratios, a fetal genotype, determination of aneuploidy, determination of aneuploidy inheritance (e.g., whether aneuploidy was maternally or paternally derived), quality control metrics, and the like.

In some embodiments, outputting is performed through an additional computer implemented software program that takes data derived from a software program and provides the data as results that are output to a user. For example, data generated by a software program such as CASAVA is input, or accessed, by a sequence analysis viewer, such as that provided by Illumina, Inc. (Sequencing Analysis Viewer User Guide). The viewer software is an application that allows for graphical representation of a sequencing analysis, quality control associated with said analysis and the like. In some embodiments, a viewing application that provides graphical output based on practicing the computational methods comprising the algorithms as described herein is installed on a sequencing instrument or computer in operational communication with a sequencing instrument (e.g., desktop computer, laptop computer, tablet computer, etc.) in a proximal location to the user (e.g., adjacent to a sequencing instrument). In some embodiments, a viewing application that provides graphical output based on practicing the computational methods comprising the algorithms as described herein is found and accessed on a computer at a distant location to the user, but is accessible by the user be remote connection, for example Internet or network connection. In some embodiments, the viewing application software is provided directly or indirectly (e.g., via externally connected hard drive, such as a solid state hard drive) onto the sequencing instrument computer.

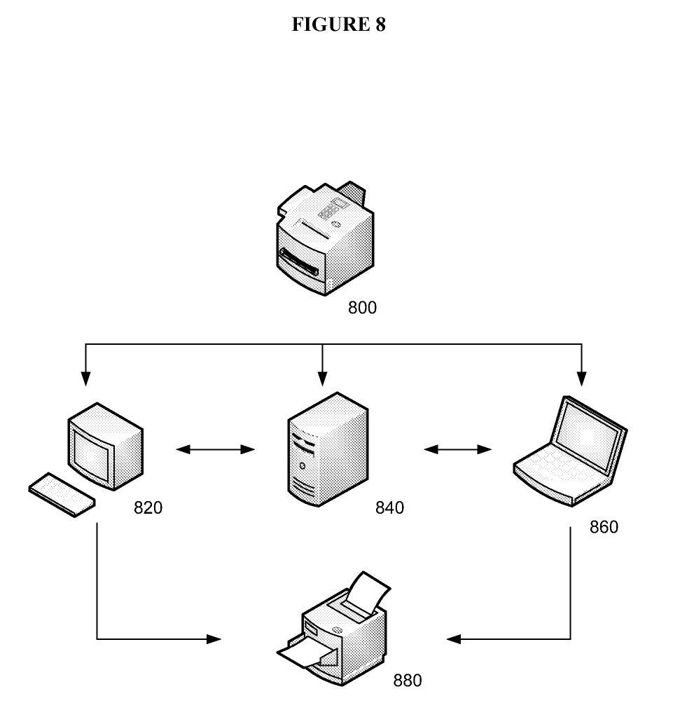

FIG. 8 illustrates an exemplary computer system that may be used to execute the computer implemented methods and systems of disclosed embodiments. FIG. 8 shows an exemplary assay instrument (800), for example a nucleic acid sequencing instrument, to which a sample is added, wherein the data generated by the instrument is computationally analyzed utilizing computer implemented methods and systems as described herein either directly or indirectly on the assay instrument. In FIG. 8, the computer implemented analysis is performed via software that is stored on, or loaded onto, an assay instrument (800) directly, or on any known computer system or storage device, for example a desktop computer (820), a laptop computer (860), or a server (840) that is operationally connected to the assay instrument, or a combination thereof. An assay instrument, desktop computer, laptop computer, or server contains a processor in operational communication with accessible memory comprising instructions for implementation of the computer implemented methods as described herein. In some embodiments, a desktop computer or a laptop computer is in operational communication with one or more computer readable storage media or devices and/or outputting devices (880). An assay instrument, desktop computer and a laptop computer may operate under a number of different computer based operational languages, such as those utilized by Apple based computer systems or PC based computer systems. An assay instrument, desktop and/or laptop computers and/or server system further provides a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry), a tablet computer (e.g., iPAD®), a hard drive, a server, a memory stick, a flash drive and the like.

A computer readable storage device or medium may be any device such as a server, a mainframe, a super computer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

In embodiments as described herein, an assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like is operated and maintained by a service provider in operational communication with an assay instrument, desktop, laptop and/or server system via an Internet connection or network connection.

In some embodiments, an assay instrument is an analysis instrument including, but not limited to, a sequencing instrument, a microarray instrument, and the like. In some embodiments, an assay instrument is a measurement instrument, including but not limited to, a scanner, a fluorescent imaging instrument, and the like for measuring an identifiable signal resulting from an assay.

In some embodiments, an assay instrument capable of generating datasets for use with computer implemented methods as described herein, as such assay instruments that comprise computer implemented systems as described herein, include but are not limited to, assay instruments as those provided by Illumina®, Inc. (HiSeq 1000, HiSeq 2000, Genome Analyzers, MiSeq, HiScan, iScan, BeadExpress systems), Applied Biosystems™ Life Technologies (ABI PRISM® Sequence detection systems, SOLiD™ System), Roche454 Life Sciences (FLX Genome Sequencer, GS Junior), Ion Torrent® Life Technologies (Personal Genome Machine sequencer) further as those described in, for example, in U.S. Pat. Nos. and patent applications 5,888,737, 6,175,002, 5,695,934, 6,140,489, 5,863,722, 2007/007991, 2009/0247414, 2010/0111768 and PCT application WO2007/123744, each of which is incorporated herein by reference in its entirety. Methods and systems disclosed herein are not necessarily limited by any particular sequencing system, as the computer implemented methods for determining the presence of a fetal chromosomal anomaly in a sample described herein are useful on any sample relevant datasets wherein alignment procedures and processes are practiced.

Computer implemented methods and systems comprising algorithms as described herein are typically incorporated into analysis software, although it is not a prerequisite for practicing the methods and systems described herein. In some embodiments, computer implemented methods and systems comprising algorithms for determining aneuploidy and/or a fetal genotype are incorporated into analysis software for analyzing sequencing datasets, for example software programs such as Pipeline, CASAVA and GenomeStudio data analysis software (Illumina®, Inc.), SOLiD™, DNASTAR® SeqMan® NGen® and Partek® Genomics Suite™ data analysis software (Life Technologies), Feature Extraction and Agilent Genomics Workbench data analysis software (Agilent Technologies), Genotyping Console™, Chromosome Analysis Suite data analysis software (Affymetrix®).

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, hardware platforms for performing computational methods as described herein comprise one or more computer systems with one or more processors. In some embodiments, smaller computers are clustered together to yield a supercomputer network (i.e., Beowulf clusters), which may be preferential under certain circumstances as a substitute for a more powerful computer system.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand alone computer systems for the purpose dealing with large amounts of data. These systems offer Perl interfaces to submit, monitor and manage large sequence analysis jobs on a cluster in serial or parallel configurations.

In some embodiments, a computer implemented method described herein is used to determine the sequence of a nucleic acid sample (e.g., a genomic DNA sample), for example nucleotide polymorphisms present in a maternal blood sample. In some embodiments, a computer implemented method is implemented upon completion of a sequencing assay. In embodiments of the present disclosure, computer implemented methods and software that comprises those methods are found loaded onto a computer system or are located on a computer readable media that is capable of being accessed and processed by a computer system. For example, computer implemented methods as described herein can read nucleic acid sequences in their entireties and input the sequences into algorithms that are implemented in a computer implemented method for determining a fetal genotype. The computed outcome can be compared to the reference genome of interest, for example, and the comparative results outputted to the user via a, for example, graphic user interface such as a computer monitor, tablet computer, and the like. However, in methods of the present application a reference sample comparison is not obligatory. As such, the computer implemented methods comprising algorithms as described herein can provide genetic information used in, for example, determining or diagnosing the presence of aneuploidy present in a fetus based on sequenceing DNA from a maternal blood sample.

### EXAMPLES

The following example is provided in order to demonstrate and further illustrate certain preferred embodiments and aspects of the present disclosure and is not to be construed as limiting the scope thereof.

Genomic DNA libraries can be generated by adding a predetermined amount of sample DNA to, for example, the Paired End Sample prep kit PE-102-1001 (ILLUMINA, Inc.) following manufacturer's protocol. Briefly, DNA fragments are generated by random shearing and conjugated to a pair of oligonucleotides in a forked adaptor configuration. The ligated products are amplified using two oligonucleotide primers, resulting in double-stranded blunt-ended products having a different adaptor sequence on either end. The libraries once generated are applied to a flowcell for cluster generation.

Clusters were formed prior to sequencing using the V3 cluster kit (ILLUMINA, Inc.) following manufacturer's instructions. Briefly, products from a DNA library preparation are denatured and single strands annealed to complementary oligonucleotides on the flow-cell surface. A new strand is copied from the original strand in an extension reaction and the original strand is removed by denaturation. The adaptor sequence of the copied strand is annealed to a surface-bound complementary oligonucleotide, forming a bridge and generating a new site for synthesis of a second strand. Multiple cycles of annealing, extension and denaturation in isothermal conditions resulted in growth of clusters, each approximately 1 μm in physical diameter.

The DNA in each cluster is linearized by cleavage within one adaptor sequence and denatured, generating single-stranded template for sequencing by synthesis (SBS) to obtain a sequence read. To perform paired-read sequencing, the products of read 1 can be removed by denaturation, the template is used to generate a bridge, the second strand is re-synthesized and the opposite strand is cleaved to provide the template for the second read.

Sequencing can be performed using the ILLUMINA, Inc. V4 SBS kit with 100 bp paired end reads on the HiSeq 2000. Briefly, DNA templates can be sequenced by repeated cycles of polymerase-directed single base extension. To ensure base-by-base nucleotide incorporation in a stepwise manner, a set of four reversible terminators, A, C, G and T each labelled with a different removable fluorophore are used. The use of modified nucleotides allows incorporation to be driven essentially to completion without risk of over-incorporation. It also enables addition of all four nucleotides simultaneously minimizing risk of misincorporation. After each cycle of incorporation, the identity of the inserted base is determined by laser-induced excitation of the fluorophores and fluorescence imaging is recorded. The fluorescent dye and linker is removed to regenerate an available group ready for the next cycle of nucleotide addition. The HiSeq sequencing instrument is designed to perform multiple cycles of sequencing chemistry and imaging to collect sequence data automatically from each cluster on the surface of each lane of an eight-lane flow cell.

For single genome read aggregation and variant calling image analysis, base calling and Phred quality scoring can be carried out using the ILLUMINA analysis pipeline. Sequence reads from those clusters whose proximity to others may result in mixed sequence data can be ignored and filtered out (purity-filtering).

For determining allele frequencies in the maternal derived genomic DNA samples, sequences can be aligned using Elandv2e from CASAVA version 1.8 (ILLUMINA, Inc.) to any reference sequence, for example the human GRCh37.1 reference sequence. The aligned reads can be aggregated and sorted into chromosomes based on alignment positions. The sorted reads can be used to call variants using Hyrax, a Bayesian caller and GROUPER. The callers are part of the standard CASAVA 1.8 distribution and can be run using used defined parameters or default parameters. To minimize false positive SNP calls the putative SNPs can be recalled using Hyrax. A candidate SNP can be called when there is complete agreement between the initial SNP call and the recall. After sequence calling, each class of variant can be annotated against the Ensembl database release e59. Further, each variant can be queried for overlapping annotated features if an investigator so desires. At each allele being sequenced the frequencies of the reference and non-reference allele can be calculated based on the total combined reads overlapping the targeted base, ratios computed and distribution of ratios evaluated for determination of aneuploidy.

A report can be generated reporting for example the sample characteristics, including the presence/absence of aneuploidy, the location of the aneuploidy, and the genotype of the fetus of the queried alleles. Additionally, the derivation of the aneuploidy can be determined and reported (e.g., whether it is maternal or paternally derived).

All publications and patents mentioned in the present application are herein incorporated by reference. Various modifications and variations of the described methods and compositions will be apparent to those skilled in the art without departing from the scope and spirit of the disclosure. Although the invention has been described in connection with specific preferred embodiments, it should be understood that the invention as claimed should not be unduly limited to such specific embodiments. Indeed, various modifications of the described embodiments that are obvious to those skilled in the relevant fields are intended to be within the scope of the following claims.

1. A method for determining fetal aneuploidy comprising:
a) obtaining the sequence of alleles at a plurality of loci in a maternally derived sample comprising maternal and fetal nucleic acids,
b) quantitating a ratio of the reference and non-reference alleles at the plurality of loci,
c) determining the distribution of the ratios of the reference and non-reference alleles at the plurality of loci, and
d) identifying the presence or absence of fetal aneuploidy in said sample based on said distribution of ratios.

2. The method of claim 1, wherein said maternally derived sample is a plasma or serum sample.

3. The method of claim 1, wherein the sequence is obtained by deep sequencing at said alleles to a mean sequence depth selected from the group consisting of at least 1000×, at least 5000× or at least 10000×.

4. The method of claim 1, wherein said reference and non-reference alleles are the same for one or more of said plurality of loci and wherein the non-reference allele is a single nucleotide polymorphism.

5. The method of claim 1, wherein said reference and non-reference alleles are different for one or more of said plurality of loci and wherein the non-reference allele is a single nucleotide polymorphism.

6. The method of claim 1, wherein said plurality of loci comprises loci on one or more of chromosome 8, 9, 13, 18, 21 and 22.

7. The method of claim 6, wherein said chromosome is chromosome 21 and wherein said fetal aneuploidy is trisomy 21.

8. The method of claim 1, wherein said quantitating a ratio comprises determining allele frequencies for the reference and non-reference alleles and the distribution of ratios comprises calculating the presence or absence of a 0.5:0.5 ratio of reference and non-reference alleles at one or more loci.

9. The method of claim 8, wherein a ratio other than a 0.5:0.5 ratio of said alleles is indicative of aneuploidy.

10. The method of claim 1, wherein the sequence is obtained using sequence by synthesis methodologies.

11. The method of claim 1, further comprising determining one or more of a fetal genotype or the paternity of the fetus.

12. The method of claim 1, wherein the percentage of fetal nucleic acids relative to total nucleic acids in the maternal sample is about 5% or less.

13. A computer implemented method for determining the presence or absence of fetal aneuploidy of claim 1 comprising:
a) quantitating the allele frequencies of reference and non-reference alleles at a plurality of loci from a nucleic acid sample comprising fetal nucleic acids,
b) computationally determining the ratio of the allele frequencies of said alleles,
c) computationally generating the distribution of ratios of the alleles, and
d) determining the presence or absence of fetal aneuploidy based on said distribution of ratios.

14. The computer implemented method of claim 13, wherein said quantitating comprises deep sequencing said nucleic acid and wherein said deep sequencing comprises determining the sequence of said alleles at a mean sequence depth selected from the group consisting of at least 1000×, at least 5000× or at least 10000×.

15. The computer implemented method of claim 13, wherein the percentage of fetal nucleic acids relative to total nucleic acids in the maternal sample is about 5% or less.

16. The computer implemented method of claim 13, further comprising outputting a prenatal diagnosis based on the presence or absence of fetal aneuploidy wherein said prenatal diagnosis comprises the presence or absence of trisomy 21.

17. The computer implemented method of claim 13, wherein said reference and non-reference alleles at a locus are the same for one or more of said loci or are different for one or more loci and wherein said non-reference allele is a single nucleotide polymorphism.

18. The computer implemented method of claim 16, further comprising outputting a fetal genotype.
